(19) United States
(12) Patent Application Publication
(10) Pub. No.: US 2005/0014142 A1
(43) Pub. Date: Jan. 20, 2005

(54) Hybridization normalization methods

(76) Inventors: Uwe Scherf, Gaithersburg, MD (US); Michael Elashoff, Gaithersburg, MD (US); Yasmin Beazer-Barclay, Gaithersburg, MD (US); Kristen J. Antonellis, Gaithersburg, MD (US); Scott A. Jelinsky, Cambridge, MD (US); Maryann Whitley, Quincy, MA (US); Eugene L. Brown, Newton, MA (US)

Correspondence Address: MORGAN LEWIS & BOCKIUS LLP, 1111 PENNSYLVANIA AVENUE NW, WASHINGTON, DC, 20004, US

(21) Appl. No.: 10/479,866

(22) PCT Filed: Jun. 6, 2002
(86) PCT No.: PCT/US02/17813

Related U.S. Application Data
(60) Provisional application No. 60/295,835, filed on Jun. 6, 2001.

(51) Int. Cl. C12Q 1/68

(52) U.S. Cl. 435/6

(57) ABSTRACT

The present invention includes methods of normalizing hybridization reactions that are designed to select normalization control genes, specifically 5′-3′-, and middle portions of the these genes, that hybridize similarly to a probe array and that produce the most consistently linear curve of hybridization signal over a range of normalization control gene segment concentrations. These methods have applicability across a broad spectrum of hybridization formats.

### RELATED APPLICATIONS

This application is related to U.S. provisional application No. 60/295,835, filed Jun. 6, 2001 and is herein incorporated by reference in its entirety.

### FIELD OF THE INVENTION

The invention relates generally to methods for normalizing hybridization reactions and optimizing the selection of normalization controls.

### BACKGROUND OF THE INVENTION

Nucleic acid hybridization-based methods have become prevalent in medical and biotechnological research and development, diagnostic testing, drug development and forensics. The reliability and utility of these nucleic acid hybridization-based methods depends on accurate and reliable methods for accounting for variations between analyses. For example, variations in hybridization conditions, label intensity, reading and detector efficiency, sample concentration and quality, background effects, and image processing effects each contribute to hybridization signal heterogeneity. Hegde et al. (2000) Biotechniques 29 (3): 548-562; Berger et al. (2000) WO 00/04188.

Normalization of hybridization procedures such as Northern blot and Dot Blot analyses has often relied on control hybridizations to housekeeping genes such as (β-actin, glyceraldehyde-3-phosphate dehydrogenase, and the transferrin receptor gene. Eickhoff et al. (1999) Nucleic Acids Research 27 (22): e33; Spiess et al. (1999) Biotechniques 26 (1): 46-50. These methods, however, generally do not provide the linearity sufficient to detect small but significant changes in transcription or gene expression. Spiess et al. (1999) Biotechniques 26 (1): 46-50. In addition, the steady state levels of many housekeeping genes are susceptible to alterations in expression levels that are dependent on cell differentiation, nutritional state, specific experimental and stimulation protocols. Eickhoff et al. (1999) Nucleic Acids Research 27 (22): e33; Spiess et al. (1999) Biotechniques 26 (1): 46-50; Hegde et al. (2000) Biotechniques 29 (3): 548-562; and Berger et al. (2000) WO 00/04188.

In addition to numerous assay-associated factors, such as variations in background, labeling, hybridization conditions and detection, characteristics of the hybridization control molecule itself, such as variations in base composition, probe length, secondary structure and ability to cross-hybridize with the probes or target nucleic acids, also contribute to the difficulty and imprecision of comparing results between analyses. The normalization of array format hybridizations has typically been conducted using full-length hybridization controls that are complementary to oligonucleotide probes contained on the array. (Affymetrix GeneChip® Expression Analysis Manual). Full-length hybridization controls, however, increase the likelihood of control-specific background effects as the normalization curves generated using full-length normalization controls may not achieve the linearity and reproducibility necessary for many of the emerging applications of array hybridization methodologies.

### SUMMARY OF THE INVENTION

The present invention is based on the surprising discovery of methods for optimizing the normalization of hybridization reactions comprising a nucleic acid sample, the method comprising the step of adding at least one normalization control gene segment to the hybridization reaction corresponding to the 3′, 5′ and middle regions of at least one normalization control gene. The normalization controls of the present invention are selected from nucleic acids that are not present in the nucleic acid sample. Preferably, the normalization controls are selected from, viral, prokaryotic or eukaryotic genes. In a preferred embodiment, the normalization control genes are selected from a Escherichia coli BioB, BioC, or BioD gene, a P1 bacteriophage cre gene, or a Bacillus subtilis dap, thr, trp, phe or lys gene.

The normalization control gene segments of the present invention are typically either DNA or RNA and may be produced by the polymerase chain reaction or cloning of the normalization control genes or segments into a vector and expression of the normalization control genes or segments in a host cell. RNA normalization control gene segments may be produced, for example, by in vitro transcription of the cloned normalization control genes or segments.

The methods of the present invention are applicable to any hybridization assay format. Preferred formats include formats where an oligonucleotide probe, complementary to the normalization control gene segments, is immobilized on a solid support such as filters, polyvinyl chloride dishes, silicon or glass beads or wafers in an array. Preferred arrays include high density or nucleic acid chip arrays. The oligonucleotide probes may be selected from nucleic acids isolated from human, non-humans, animals, microorganisms, bacteria, fungi, plants, and nucleic acids isolated from specific normal or diseased tissue.

The nucleic acid samples compatible with the methods of the instant invention include pooled nucleic acid samples, genomic DNA, cDNA, cRNA, mRNA, and polyA RNA.

The normalization control gene segments of the instant invention are selected by a method that comprises determining the non-specific cross-hybridization of the nucleic acid sample to the normalization control gene segments, wherein the normalization control gene segments that do not substantially cross-hybridize are selected. In another embodiment, the normalization controls of the present invention are selected by a method comprising analyzing a series of hybridization reactions, wherein each hybridization reaction of the series contains an increased concentration of the normalization control gene segment, and wherein the normalization control gene segments that produce the most consistently linear curve of hybridization signal over a range of normalization control gene segment concentrations are selected.

In a preferred embodiment, the methods of normalizing a hybridization reaction of the present invention comprise the steps of:
a) providing a normalization control comprising one or more normalization control gene segments, wherein said normalization control gene segments are mixed with the nucleic acid sample, and wherein said normalization control gene segments are prepared by a method comprising:
   i) selecting one or more candidate normalization control genes;
   ii) segmenting the candidate normalization control genes into 5′-, middle-, and 3′-segments, thereby producing candidate normalization control gene segments;
   iii) hybridizing said candidate normalization control gene segments to an oligonucleotide probe in the presence and absence of the nucleic acid sample;
   iv) determining the non-specific cross-hybridization of candidate normalization control gene segments to said oligonucleotide probe by determining the hybridization of candidate normalization control gene segments to probes other than those complementary to the candidate normalization control gene segments;
   v) repeating step (iii) at various concentrations of candidate normalization control gene segments; and
   vi) identifying and selecting those candidate normalization control gene segments that do not substantially cross-hybridize to said oligonucleotide probe.

In a more preferred embodiment, the methods of normalizing a hybridization reaction of the present invention comprise steps wherein the normalization control gene segments are prepared by method further comprising the following steps:
a) preparing individual mixtures of nucleic acid samples and candidate normalization control gene segments wherein each individual mixture contains a different concentration of the candidate normalization control gene segments identified in step (vi);
b) hybridizing a mixture of step (a) to an oligonucleotide probe;
c) repeating step (b) with mixtures containing different concentrations of candidate normalization control gene segments;
d) identifying the candidate normalization control gene segments that produce the most consistently linear hybridization response over a range of candidate normalization control gene segment concentrations by measuring the hybridization of said candidate normalization control gene segments to oligonucleotide probes that are complementary to the normalization control gene segments over a range of candidate normalization control gene segment concentrations; and
e) producing a solution or composition containing one or more of the candidate normalization control gene segments of step (d) over a concentration range sufficient to produce a linear normalization curve.

In the most preferred embodiment, the methods of the present invention further comprise the steps of hybridizing a mixture of said nucleic acid sample and the solution of step (e) to said array, and quantifying the hybridization of said target or pool of nucleic acid sample to said array.

The methods of the present invention also contemplate using normalization control gene segments that are labeled with either a fluorescent, chemiluminescent, bioluminescent, colorimetric, or a light scattering label.

In another embodiment, the methods of the present invention further comprise the step of fragmenting the normalization control gene segments prior to use.

### BRIEF DESCRIPTION OF THE DRAWINGS

FIG. 1. Standard curves for each normalization control gene segment hybridized to a GeneChip® at concentrations ranging from 0.5-100 pM.

FIG. 2. Standard curves generated from hybridization of normalization control gene cocktail 831, 849, and 7211 to various GeneChips®.

FIG. 3. Standard curve generated from hybridization of normalization control gene cocktail 7211, which contains BioB3′ at 75 pM and BioD3′ at 100 pM, to various GeneChips®.

### DETAILED DESCRIPTION

The present Inventors have developed methods of normalizing hybridization reactions that are designed to select normalization control genes, specifically the 5′-, 3′-, and middle-portions of the these genes, that hybridize to a probe array and produce the most consistently linear hybridization signal over a range of normalization control gene segment concentrations. These methods have applicability across a broad spectrum of hybridization formats. Although any nucleic acid may serve as a normalization control, a careful analysis of the specific characteristics of any given normalization control will enable optimization of the linearity of the normalization control hybridization signal, thereby increasing both the accuracy and precision of the analyses. The normalization controls of the present invention may be selected from a variety of sources and different coding and non-coding regions. The identity of the normalization control ultimately selected will depend on the specific application and hybridization format in which the control will be used. The present invention is applicable to any normalization control or set of normalization controls that can be selected and prepared, by the methods of the present invention, for use in hybridization reactions of any format.

A. Hybridization Controls.

In addition to specific oligonucleotide probes which bind the nucleic acid sample, a hybridization format may contain one or more control probes. The control probes fall into three categories referred to herein as: (1) normalization control probes; (2) expression level control probes; and (3) mismatch control probes.

As used herein, “normalization controls” are polynucleotides, oligonucleotides or other nucleic acids that are added to a nucleic acid sample and include “normalization control genes” and “control gene segments”. As used herein, “normalization control probes” are oligonucleotides or other nucleic acid probes that are complementary to the normalization control genes or normalization control gene segments and are used to detect or quantitate the normalization control genes or normalization control gene segments in a nucleic acid sample.

As used herein, “normalization control gene segment(s)” is a portion of the “normalization control gene(s)”. Preferably, a normalization control gene segment comprises the 5′-, 3′- or middle-portion of the “normalization control gene”.

The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity), read from all other probes in the array, are divided by the signal from the control probes, thereby normalizing the measurements.

As used herein, “expression level controls” are nucleic acids that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to the β-actin gene, the transferrin receptor gene, the glyceraldehyde-3-phosphate dehydrogenase gene (GAPDH), and the like.

As used herein, “mismatch control” refers to an oligonucleotide whose sequence is deliberately selected not to be perfectly complementary to a particular oligonucleotide probe. For each mismatch (MM) probe in a high-density array there typically exists a corresponding perfect match (PM) probe that is perfectly complementary to the same particular mismatch control sequence. The mismatch may comprise one or more bases.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the mismatch control probe under the test hybridization conditions. Mismatch controls thus provide a control for non-specific binding or cross-hybridization of the control sequence to an oligonucleotide probe other than the one to which the mismatch control is directed. Mismatch controls also indicate whether a hybridization is specific or not.

As used herein, “perfect match probe” refers to a probe that has a sequence that is perfectly complementary to a particular control sequence. The perfect match probe is typically perfectly complementary to a portion (subsequence) of the control sequence. The perfect match probe can be a “test probe” a “normalization control” probe, an expression level control probe and the like. A perfect match control, however, is distinguished from a “mismatch control.”

1. Selection of Normalization Controls.

The nucleic acids of the normalization-controls of the present invention can be obtained from any source. A preferred source is animal nucleic acids, and in some formats a more preferred source is human nucleic acids. Plant nucleic acids, and microbial nucleic acids, specifically including bacterial and fungal nucleic acids, are also preferred sources of normalization control nucleic acids. Although any nucleic acid may be utilized as a normalization control for any hybridization format, the normalization control for a particular hybridization reaction is preferably: 1) neither related to the family of sequences present in the nucleic acid sample nor their corresponding oligomeric probes; 2) identical to the sequence or subsequence of a normalization control probe that is included in the hybridization assay; and 3) easily synthesized or prepared.

As used herein, normalization control gene nucleic acids that meet the above criteria for a particular hybridization reaction are referred to as “candidate normalization control genes.” Following identification, the “candidate normalization control genes” are then segmented. In a preferred embodiment, these “normalization control gene segments” correspond to between about 95% and 75% of the normalization control gene; preferably between about 75% and 50% of the normalization control gene, and more preferably between about 50% and 25% or between about 25% and 5% of the normalization control gene. In another embodiment, the normalization control gene segments correspond to the 5′-, middle-, and 3′-regions of the normalization control gene. As used herein, “5′-region” of the normalization control gene refers to the about one-third of the normalization control gene that begins at the 5′-end of either the sense or anti-sense strand of the normalization control gene. As used herein, “middle-region” of the normalization control gene refers to the middle about one-third of either the sense or anti-sense strand of the normalization control gene. As used herein, “3′-region” of the normalization control gene refers to the about one-third of the normalization control gene that begins at the 3′-end of either the sense or anti-sense strand of the normalization control gene.

The cross-hybridization of the candidate normalization control gene segments is analyzed by comparing the hybridization of the normalization control gene segments in the presence and absence of nucleic acid sample. As used herein, the terms “cross-hybridize(s)” and “cross-hybridization” refer to hybridization resulting from non-specific binding, or other interactions, between the labeled normalization control gene segment(s) and components of the hybridization reaction other than the normalization control probe(s) that is complementary to the normalization control gene segment(s) (e.g., the oligonucleotide probes, other non-complementary control probes, the substrate or matrix of the particular hybridization reaction, nucleic acid sample, etc.).

As used herein, “background” refers to signals associated with non-specific binding (cross-hybridization). In addition to cross-hybridization, background may also be produced by intrinsic fluorescence of the hybridization format components themselves. A single background signal can be calculated for the entire format, or a different background signal may be calculated for each nucleic acid sample or normalization control gene segment. In a preferred embodiment, background is calculated as the average hybridization signal intensity for the lowest 5% to 10% of the probes in an array, or, where a different background signal is calculated for each nucleic acid sample or normalization control gene segment, for the lowest 5% to 10% of the probes for each sample. Of course, one of skill in the art will appreciate that where the probes to a particular sample or normalization control gene segment hybridize well, and thus, appear to specifically bind to a nucleic acid sample or normalization control gene segment, they should not be used in a background signal calculation. Alternatively, background may be calculated as the average hybridization signal intensity produced by hybridization to probes that are not complementary to any sequence found in the nucleic acid sample or normalization control gene segment (e.g., probes directed to nucleic acids of the opposite sense or to genes not found in the sample, such as bacterial genes where the sample is mammalian nucleic acids). In nucleic acid array formats, for example, background can be calculated as the average signal intensity produced by regions of the array that lack any probes at all.

As used herein, normalization control genes or normalization control gene segments that are “complementary” to one or more of the oligonucleotide probes used in the hybridization formats described herein, refers to normalization control genes or normalization control gene segments that are capable of hybridizing under stringent conditions to at least part of the oligonucleotide probe. Such hybridizable normalization control genes or normalization control gene segments will typically exhibit at least about 75% sequence identity at the nucleotide level to said probes, preferably about 80% or 85% sequence identity or more preferably about 90% or 95% or more sequence identity to said probes.

“Bind(s) substantially” refers to complementary hybridization between an oligonucleotide probe and a nucleic acid sample or normalization control gene segment and embraces minor mismatches that can be accommodated by reducing the stringency of the hybridization media to achieve the desired detection of the nucleic acid sample.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

In order to determine the optimal concentration for use with each individual normalization control gene segment, a nucleic acid sample is mixed with one normalization control gene segment, at a particular concentration of the normalization control gene segment, and hybridized to the oligonucleotide probes according to the procedures described herein. Each normalization control gene segment is analyzed over a range of concentrations which, for example, may include about 0.1 pm to about 50 nM or include about 0.5 pM, 0.75 pM, 1.0 pM, 1.5 pM, 2 pM, 3 pM, 5 pM, 12.5 pM, 25 pM, 50 pM, 75 pM, 100 pM and 150 pM. The median intensity of the normalization control gene segments bound at each concentration is plotted so that the normalization control gene segments that hybridize to the probes and produce the most consistently linear curve of hybridization signal over a range of normalization control gene segment concentrations are selected. A linear correlation is any relationship between two variables (i.e. normalization control gene segment concentration and hybridization signal) such that a graphical plot of one variable against the other produces a approximately straight line. As used herein, “linear coefficient” refers to the degree to which the relationship of one variable to another produces a line with a slope equal to about 1.00. As used herein, “linear curve” refers to a line that has a linear coefficient of r=about 0.980 to about 1.000. As used herein, “consistently linear curve” refers to a series of linear curves, derived from a series of analyses of a nucleic acid sample, using the normalization controls of the present invention, wherein the linear curves generated from plotting the hybridization signal versus the concentration of the normalization control have linear coefficients between r about 0.985 and r=about 1.000.

2. Preparation of Normalization Controls.

Nucleic acids to be used as normalization control genes may be obtained from a variety of natural sources such as organisms, organs, tissues and cells. The sequences of known genes are in the public databases. The sequences of the genes in GenBank are expressly incorporated by reference. The complete genomes of several organisms are available at the National Center for Biotechnology Information (see, http://www.ncbi.nlm.nih.gov/Entrez/Genome/org.html.). Normalization control genes that are based on the sequences of these genes, for example, may be prepared by any commonly available method or obtained from the American Type Culture Collection (ATCC), Manassas, Va., for example, or other commercial sources. Normalization control genes of the present invention include single-stranded or double-stranded nucleic acid molecules, including RNA, DNA, cRNA and cDNA.

Sources of normalization control gene nucleic acids include prokaryotic cells, such as the bacterial cells of species of the genera Escherichia, Bacillus, Serratia, Salmonella, Neisseria, Treponemia, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces. Sources of normalization control genes also include eukaryotic cells such as fungi, especially yeast, plants, protozoans, parasites, animals, insects, especially Drosophila, nematodes, especially Caenorhabditis elegans, and mammals, including humans.

The candidate normalization control genes can be digested with any commercially available restriction endonuclease or other cleaving agent, under conditions sufficient to produce a 5′-, middle-, and 3′-portion of a normalization control gene. Following isolation and purification, these resultant normalization control gene segments can be used directly, amplified by PCR methods or amplified by replication or expression from a vector. PCR techniques comprise the hybridization (annealing) of two primer oligonucleotides to a template nucleic acid and elongation of the oligonucleotide primers by a thermostable polymerase. Multiple cycles of polymerization, denaturation and annealing result in amplification of the template nucleic acid. (See, Mullis et al. (1987) Meth. Enzymol. 155: 335-350; U.S. Pat. No. 4,683,195; U.S. Pat. No. 4,683,202).

RNA or DNA can be produced by in vitro transcription from a template polynucleotide, using commercially available reagents and kits from New England Biolabs, Beverly, Mass.; Invitrogen Corporation, San Diego, Calif., or Ambion, Incorporated, Austin, Tex. To utilize in vitro transcription reactions, the desired template is constructed by operably linking a target polynucleotide sequence to a promoter that is recognized by polymerase to produce either DNA or RNA. Examples of promoters include: the T3 phage promoter; the T7 phage promoter; and the SP6 phage promoter. If the Ambion, Inc. MEGAscript™ T7 kit (Cat. No. 1334) is used, the polynucleotide sequence is operably linked to a T7 phage promoter.

Normalization control gene segments produced by the polymerase chain reaction (PCR), direct synthesis or restriction endonuclease digestion can be amplified by placing the normalization control gene segment in a vector according to established protocols. Sambrook et al (1989) Molecular Cloning: A Laboratory Manual, Second Edition; DNA Cloning, Vols. I and II (D. N. Glover ed. 1985); Perbal (1984) A Practical Guide to Molecular Cloning; Gene Transfer Vectors for Mammalian Cells(J. H. Miller et al. eds. (1987) Cold Spring Harbor Laboratory, Cold Spring Harbor, N.Y.); Scopes, Protein Purification: Principles and Practice (2^{nd} ed., Springer-Verlag); PCR: A Practical Approach McPherson et al. eds. (1991) IRL Press. The resultant vectors can be used to transform bacterial cells by established protocols. See e.g., Sambrook et al. The transformed bacterial cells can be cultured according to established protocols. See e.g., Sambrook et al. The plasmid DNA from the overnight cultures can be isolated using QIAGEN plasmid kits and other standard procedures. See e.g., Sambrook et al. The isolated plasmid DNA is digested with an appropriate restriction endonuclease according to the manufacturer's protocols. Digestion of the isolated plasmid can be monitored by gel electrophoresis in a 1% agarose gel.

Normalization control genes and normalization control gene segments (i.e., synthetic oligo- and polynucleotides) can easily be synthesized by chemical techniques, for example, the phosphotriester method of Matteucci, et al ((1981) J Am. Chem. Soc. 103: 3185-3191) or using automated synthesis methods. In addition, larger nucleic acids can readily be prepared by well known methods, such as synthesis of a group of oligonucleotides that define various modular segments of the normalization control genes and normalization control gene segments, followed by ligation of oligonucleotides to build the complete nucleic acid molecule.

The present invention further provides recombinant nucleic acid molecules that encode the normalization control genes and normalization control gene segments. As used herein, a “recombinant nucleic acid molecule” refers to a nucleic acid molecule that has been subjected to molecular manipulation in vitro. Methods for generating recombinant DNA (rDNA) molecules are well known in the art. See e.g., Sambrook et al. (1989); Perbal (1984); and Scopes (1991). In the preferred recombinant nucleic acid molecules, a nucleotide sequence that encodes a normalization control gene or a normalization control gene segment is operably linked to one or more expression control sequences and/or vector sequences.

The choice of vector and/or expression control sequences to which the normalization control genie or normalization control gene segment is operably linked depends directly, as is well known in the art, on the functional properties desired (e.g., the host cell to be transformed). A vector contemplated by the present invention is at least capable of directing the replication or amplification, of the nucleotide sequence encoding the normalization control gene or normalization control gene segment.

In one embodiment, the vector containing a normalization control gene or normalization control gene segment will include a prokaryotic replicon, i.e., a DNA sequence having the ability to direct autonomous replication and maintenance of the recombinant DNA molecule intrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin (Amp) or tetracycline (Tet).

Vectors that include a prokaryotic replicon can further include a prokaryotic or viral promoter capable of directing the expression (transcription) of the normalization control gene or normalization control gene segment in a bacterial host cell, such as E. coli. A promoter is a control element formed by a nucleotide sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from Biorad Laboratories (Richmond, Calif.), pPL and pKK23 available from Pharmacia, Piscataway, N. J.

Expression vectors compatible with eukaryotic cells, preferably those compatible with vertebrate cells, can also be used to express nucleic acid molecules that contain a nucleotide sequence that encodes a normalization control gene or normalization control gene segment. Eukaryotic cell expression vectors are well known in the art and are available from several commercial sources. Typically, such vectors provide convenient restriction sites for insertion of the desired nucleic acid segment. Typical of such vectors are pSVL and pKSV-10 (Pharmacia), pBPV-1/pML2d (International Biotechnologies, Inc.), pTDT1 (ATCC, #31255), the vector pCDM8 described herein, and other like eukaryotic expression vectors.

Eukaryotic cell expression vectors used to construct the recombinant molecules of the present invention may further include a selectable marker that is effective in a eukaryotic cell, preferably a drug resistance selection marker. A preferred drug resistance marker is the gene whose expression results in neomycin resistance, i.e., the neomycin phosphotransferase (neo) gene. Southern et al., J. Mol. Anal. Genet. (1982) 1:327-341. Alternatively, the selectable marker can be present on a separate plasmid, and the two vectors are introduced by cotransfection of the host cell, and selected by culturing in the presence of the appropriate drug for the selectable marker.

The present invention farther provides host cells transformed with a nucleic acid molecule that encodes a normalization control gene or normalization control gene segment of the present invention. The host cell can be either prokaryotic or eukaryotic. Eukaryotic cells useful for replication of a normalization control gene or normalization control gene segment are not limited, so long as the cell line is compatible with cell culture methods and compatible with the propagation of the expression vector and expression of the normalization control genes or normalization control gene segments. Preferred eukaryotic host cells include, but are not limited to, yeast, insect and mammalian cells, preferably vertebrate cells such as those from a mouse, rat, monkey or human fibroblastic cell line.

Transformation of appropriate cell hosts with nucleic acid molecules encoding a normalization control gene or normalization control gene segment of the present invention is accomplished by well known methods that typically depend on the type of vector and host system employed. With regard to transformation of prokaryotic host cells, electroporation and salt treatment methods are typically employed. See e.g., Cohen et al., Proc Natl Acad Sci USA (1972) 69:2110; Maniatis et al., Molecular Cloning. A Laboratory Manual, Cold Spring Harbor Laboratory, Cold Spring Harbor, N.Y. (1982); Sambrook et al. (1989); Perbal (1984); and Scopes (1991). With regard to transformation of vertebrate cells with vectors containing rDNAs, electroporation, cationic lipid or salt treatment methods are typically employed. See, for example, Graham et al., Virology (1973) 52:456; Wigler et al, Proc. Natl. Acad. Sci. U.S.A. (1979) 76:1373-76.

Successfully transformed cells, i.e., cells that contain a nucleic acid molecule encoding the normalization control gene or normalization control gene segment of the present invention, can be identified by well known techniques. For example, cells resulting from the introduction of a nucleic acid molecule of the present invention can be cloned to produce single colonies. Cells from those colonies can be harvested, lysed and their nucleic acids content examined for the presence of the recombinant molecule using a method such as that described by Southern, J. Mol. Biol. (1975) 98:503, or Berent et al., Biotech. (1985) 3:208. The present invention further provides methods for producing a normalization control gene or normalization control gene segment. In general terms, the production of a recombinant normalization control gene or normalization control gene segment typically involves the following steps.

First, a nucleic acid molecule is obtained that encodes a normalization control gene or normalization control gene segment. Said nucleic acid molecule is then preferably placed in an operable linkage with suitable control sequences, as described above. The expression unit is used to transform a suitable host and the transformed host is cultured under conditions that allow the production of the normalization control gene or normalization control gene segment. Optionally, the rDNA molecule is isolated from the medium or from the cells; recovery and purification of the normalization control gene or normalization control gene segment may not be necessary in some instances where some impurities may be tolerated.

Each of the foregoing steps can be done in a variety of ways. For example, the desired sequences may be obtained from genomic fragments and used directly in an appropriate host. The construction of vectors that are operable in a variety of hosts is accomplished using an appropriate combination of replicons and control sequences. The control sequences, vectors, and transformation methods are dependent on the type of host cell used to express the gene and were discussed in detail earlier. A skilled artisan can readily adapt any host system known in the art for use with the nucleotide sequences described herein to produce the normalization control genes or normalization control gene segments of the present invention.

The individual normalization control gene segments can be fragmented by chemical, mechanical or enzymatic methods that are well known in the art. See, e.g., Sambrook et al. (1989). Preferably, normalization control gene segment RNA is fragmented by magnesium ion-induced hydrolysis at alkaline pH and elevated temperature. Most preferably, RNA is fragmented in fragmentation buffer (40 mM Tris-acetate (pH 8.1); 100 mM potassium acetate; 30 mM magnesium chloride) at 95° C. between 25 and 50 minutes.

The hybridized nucleic acids are typically detected by detecting one or more labels attached to the sample nucleic acids and the normalization controls. The available labels include but are not limited to: radioactive isotopes; fluorescent labels, such as fluorescein isothiocyanate, Texas red, rhodamine, fluorescein-12-deoxycytosine triphosphate, lissamine-5-deoxycytosine triphosphate, and the lice; polypeptides that are detectable by antibodies; biotin that is detectable by labeled avidin; chemiluminescent labels; enzymes; substrates; cofactors; magnetic particles; heavy metal atoms; and spectroscopic labels. The labels may be incorporated by any of a number of means well known to those of skill in the art. (See e.g., Lockhart et al., (1999) WO 99/32660; U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149; and U.S. Pat. No. 4,366,241).

The labels can be incorporated either during synthesis of the normalization control genes or normalization control gene segments or after synthesis of the normalization control genes or normalization control gene segments.

B. Assay or Hybridization Formats.

The present invention may be practiced with any hybridization assay format, including solution-based and solid support-based assay formats. As used herein, “hybridization assay format(s)” refer to the organization of the oligonucleotide probes relative to the nucleic acid sample. The hybridization assay formats of the present invention, for example, include assays where the nucleic acid sample is labeled with one or more detectable labels, assays where the probes are labeled with one or more detectable labels, and assays where the sample or the probes are immobilized. Hybridization assay formats include but are not limited to: Northern blots, Southern blots, dot blots, solution-based assays, branched-DNA assays, microarrays and biochips.

As used herein a “probe” or “oligonucleotide probe” is defined as a nucleic acid, capable of binding to a nucleic acid sample or normalization control gene segment of complementary sequence through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages. The oligonucleotide probes comprising the oligonucleotide arrays can be obtained from any source. A preferred source is animal nucleic acids, and a more preferred source is human nucleic acids. Plant nucleic acids, and microbial nucleic acids, specifically including bacterial and fungal nucleic acids, are also preferred sources of oligonucleotide probes. In another embodiment of the invention tissue specific nucleic acids and disease-specific nucleic acids are the preferred sources of oligonucleotide probes.

Any solid surface to which oligonucleotides or nucleic acid sample can be bound, either directly or indirectly, either covalently or non-covalently, can be used. For example, solid supports for various hybridization assay formats can be filters, polyvinyl chloride dishes, silicon or glass based chips, etc. Glass-based solid supports, for example, are widely available, as well as associated hybridization protocols. (See, e.g., Beattie, WO 95/11755).

A preferred solid support is a high density array or DNA chip. This contains an oligonucleotide probe of a particular nucleotide sequence at a particular location on the array. Each particular location may contain more than one molecule of the probe, but each molecule within the particular location has an identical sequence. Such particular locations are termed features. There may be, for example, 2, 10, 100, 1000 to 10,000; 100,000 or 400,000 such features on a single solid support. The solid support, or more specifically, the area wherein the probes are attached, may be on the order of a square centimeter.

1. Dot Blots.

The normalization controls and methods of the present invention may be utilized in numerous hybridization formats such as dot blots, dipstick, branched DNA sandwich and ELISA assays. Dot blot hybridization assays provide a convenient and efficient method of rapidly analyzing nucleic acid samples in a sensitive manner. Dot blots are generally as sensitive as enzyme-linked immunoassays. Dot blot hybridization analyses are well known in the art and detailed methods of conducting and optimizing these assays are detailed in U.S. Pat. Nos. 6,130,042 and 6,129,828, and Tkatchenko et al. (2000) Biochiminca et Biophysica Acta 1500: 17-30. Specifically, labeled or unlabeled nucleic acid sample is denatured and bound to a membrane (i.e. nitrocellulose), and is then contacted with unlabeled or labeled oligonucleotide probes. Buffer and temperature conditions can be adjusted to vary the degree of identity between the oligonucleotide probes and nucleic acid sample necessary for hybridization.

Several modifications of the basic Dot blot hybridization format have been devised. For example, Reverse Dot blot analyses employ the same strategy as the Dot blot method, except that the oligonucleotide probes are bound to the membrane and the nucleic acid sample is applied and hybridized to the bound probes. Similarly, the Dot blot hybridization format can be modified to include formats where either the nucleic acid sample or the oligonucleotide probe is applied to microtiter plates, micorbeads or other solid substrates. Each of these variations on the basic Dot blot hybridization format may be used to detect and analyze any nucleic acid sample, including allelic variation between individuals, detection of single nucleotide polymorphisms (SNPs), genotyping and genetic mapping, gene expression and differential gene expression between normal and diseased (i.e. pathological or metastatic) tissues or cells.

2. Membrane-Based Formats.

Although each membrane-based format is essentially a variation of the Dot blot hybridization format, several types of these formats are preferred. Specifically, the methods of the present invention may be used in Northern and Southern blot hybridization assays. Although the methods of the present invention are generally used in quantitative nucleic acid hybridization assays, these methods may be used in qualitative or semi-quantitative assays such as Southern blots, in order to facilitate comparison of blots. Southern blot hybridization, for example, involves cleavage of either genomic or cDNA with restriction endonucleases followed by separation of the resultant fragments on a polyacrylamide or agarose gel and transfer of the nucleic acid fragments to a membrane filter. Labeled oligonucleotide probes are then hybridized to the membrane-bound nucleic acid fragments. In addition, intact cDNA molecules may also be used, separated by electrophoresis, transferred to a membrane and analyzed by hybridization to labeled probes. Northern analyses, similarly, are conducted on nucleic acids, either intact or fragmented, that are bound to a membrane. The nucleic acids in Northern analyses, however, are generally RNA.

3. Arrays.

High-throughput analysis of genetic sequences has been accomplished by the development of oligonucleotide, and micro-array technology. Oligonucleotide probe arrays can be made and used according to any techniques known in the art (see for example, Lockhart et al., (1996) Nat. Biotechnol. 14, 1675-1680; McGall et al., (1996) Proc. Nat. Acad. Sci. USA 93, 13555-13460). Array formats may be used to detect and analyze allelic variation between individuals, detection of single nucleotide polymorphisms (SNPs), genotyping and genetic mapping, gene expression and differential gene expression between normal and diseased (i.e. pathological or metastatic) tissues or cells. Such probe arrays may contain at least two or more oligonucleotides that are complementary to or hybridize to one or more of the nucleic acids of the nucleic acid sample and/or the normalization control genes or normalization control gene segments. Such arrays may also contain oligonucleotides that are complementary or hybridize to at least 2, 3, 4, 5, 6, 7, 8, 9, 10, 20, 30, 50, 70 or more of the nucleic acids of the nucleic acid sample.

Oligonucleotide probes for assaying the tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases longer probes of at least 30, 40, or 50 nucleotides will be desirable. The oligonucleotide probes of high density array chips include oligonucleotides that range from about 5 to about 45 or 5 to about 500 nucleotides, more preferably from about 10 to about 40 nucleotides and most preferably from about 15 to about 40 nucleotides in length. In other particularly preferred embodiments the probes are 20 or 25 nucleotides in length. In another preferred embodiment, probes are double or single strand DNA sequences. DNA sequences are isolated or cloned from natural sources or amplified from natural sources using natural nucleic acid as templates. These probes have sequences complementary to particular subsequences of the nucleic acid sample and/or normalization control gene segments. Thus, the oligonucleotide probes are capable of specifically hybridizing to the nucleic acid sample and/or the normalization control gene segments.

One of skill in the art will appreciate that an enormous number of array designs are suitable for the practice of this invention. The high density array will typically include a number of probes that specifically hybridize to the sequences of interest. (See WO 99/32660 for methods of producing probes for a given gene or genes.) Assays and methods of the invention may utilize available formats to simultaneously screen at least about 100, preferably about 1000, more preferably about 10,000 and most preferably about 1,000,000 different nucleic acid hybridizations.

The methods of this invention are also applicable to commercially available oligonucleotide arrays. A preferred oligonucleotide array may be selected from the Affymetrix, Inc. GeneChip® series of arrays which include the GeneChip® Human Genome U95 Set, GeneChip® Hu35K Set, GeneChip®, HuGeneFL Array, GeneChip® Human Cancer G 110 Array, GeneChip® Rat Genome U34 Set, GeneChip® Mu19K Set, GeneChip® Mu11K Set, GeneChip® Yeast Genome S98 Array, GeneChip® E. coli Genome Array, GeneChip® Arabidopsis Genome Array, GeneChip® HuSNP™ Probe Array, GeneChip® GenFleX™ Tag Array, GeneChip® HIV PRT Plus Probe Array, GeneChip® P53 Probe Array, GeneChip®, and the CYP450 Probe Array. In another embodiment, an oligonucleotide array may be selected from the Incyte Pharmaceuticals, Inc. GEM™ series of arrays which includes the UniGEM™ V 2.0, Human Genome GEM 1, Human Genome GEM 2, Human Genome GEM 3, Human Genome GEM 4, Human Genome GEM 5, LifeGEM™ 1 Cancer/Signal Peptide, LifeGEM 2 Inflammation/Blood, Mouse GEM 1 Rat GEM 1 Liver/Kidney, Rat GEM 2 Central Nervous System, Rat GEM 3 Liver/Kidney, S. aureus GEM 1, C. albicans GEM 1, and Arabidopsis GEM 1.

Methods of data collection, image processing and data processing are well-known in the art. Hegde et al. (2000) Biotechniques 29 (3): 548-562; Winzeller et al. (1999) Meth. Enzymol 306 (1): 3-18; Tkatchenko et al. (2000) Biochimica et Biophysica Acta 1500: 17-30; Berger et al. (2000) WO 00/04188; Schuchhardt et al. (2000) Nucleic Acids Research 28 (10): e47; Eickhoff et al. (1999) Nucleic Acids Research 27 (22): e33. Micro-array data analysis and image processing software packages and protocols are available from BioDiscovery (http://www.biodiscovery.com/) Silicon Graphics (http://www.sigenetics.com) Spotfire (http://www.spotfire.com/), Stanford University (http://rana.Stanford.EDU/software/), National Human Genome Research Institute (http://www.nhgri.nih.gov/DIR/LCG/15K/HTML/img_analysis.html) and TIGR (http://www.tigr.org/softlab/). Micro-arrays can be scanned using numerous commercially available detectors and scanners, such as the ScanArray® 3000 (GSI Lumonics, Watertown, Mass., USA), for example.

C. Hybridization.

As used herein, “nucleic acid hybridization” simply involves contacting a probe and nucleic acid sample under conditions where the probe and its complementary target can form stable hybrid duplexes through complementary base pairing (see Lockhart et al., (1999) WO 99/32660). The nucleic acids that do not form hybrid duplexes are then washed away leaving the hybridized nucleic acids to be detected, typically through detection of an attached detectable label.

It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPE-T at 37° C. to 50° C. until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

As used herein, the term “stringent conditions” refers to conditions under which a probe will hybridize to a complementary nucleic acid sample or normalization control gene segment, but with only insubstantial hybridization to other sequences. Stringent conditions are sequence-dependent and will be different under different circumstances. Longer sequences hybridize specifically at higher temperatures. Generally, stringent conditions are selected to be about 5° C. lower than the thermal melting point (Tₘ) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotide). Stringent conditions may also be achieved with the addition of destabilizing agents such as formamide,

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above that the hybridization pattern is not appreciably altered and which provides adequate signal for the particular oligonucleotide probes of interest.

The “percentage of sequence identity” or “sequence identity”is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical residue (e.g., nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Homology or identity is determined by BLAST (Basic Local Alignment Search Tool) analysis using the algorithm employed by the programs blastp, blastn, blastx, tblastn and tblastx (Karlin et al., (1990) Proc. Natl. Acad. Sci. USA 87, 2264-2268 and Altschul, (1993) J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (1994) Nature Genet. 6, 119-129) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) Proc. Natl. Acad. Sci. USA 89, 10915-10919, fully incorporated by reference). Four blastn parameters were adjusted as follows: Q=10 (gap creation penalty); R=110 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

D. Preparation of Nucleic Acid Samples.

As used herein, “nucleic acid sample” refers to any nucleic acid or pooled nucleic acid isolated from any source. A preferred nucleic acid sample contains genomic DNA or cDNA. A more preferred embodiment contains mRNA, cRNA, or polyA-RNA. The nucleic acid sample may be cloned or not and the nucleic acid may be amplified or not. The cloning itself does not appear to bias the representation of genes within a population. However, it may be preferable to use polyA-RNA as a source, as it can be used with less processing steps. As used herein, “nucleic acid sample” also refers to any nucleic acid of any origin that is applied to a partially or fully complementary nucleic acid(s), oligonucleotide(s), or oligonucleotide probe(s) in a hybridization reaction.

As is apparent to one of ordinary skill in the art, nucleic acid samples used in the methods of the present invention may be prepared by any available method or process. Methods of isolating total mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes. Part I Theory and Nucleic Acid Preparation, Tijssen, (1993) (editor) Elsevier Press. Such samples include RNA samples, but also include cDNA synthesized from a mRNA sample isolated from a cell or tissue of interest. Such samples also include DNA amplified from cDNA or genomic DNA, and RNA produced by in vitro transcription of the amplified DNA (cRNA). One of slcill in the art would appreciate that it is desirable to inhibit or destroy RNase present in homogenates before homogenates can be used.

As used herein, “biological samples” refer to any biological tissue or fluid or cells from any organism as well as cells raised in vitro, such as cell lines and tissue culture cells. Frequently, the sample will be a “clinical sample” which is a sample derived from a patient. Typical clinical samples include, but are not limited to, sputum, blood, blood-cells (e.g., white cells), serum, plasma, spinal fluid, semen, lymph, tissue or fine needle biopsy samples, tumors, organs, urine, peritoneal fluid, and pleural fluid, or cells therefrom. Biological samples may also include sections of tissues, such as frozen sections or formalin fixed sections taken for histological purposes.

Tissue samples, following homogenization, and isolated cells are lysed by conventional methods that disrupt the cells and inactivate ribonucleases (RNase) present in the sample. For example, RNase is commonly inactivated by the addition of 4M guanidinium thiocyanate and β-mercaptoethanol. Inactivation of RNase by such solutions allow for isolation of intact RNA from cells and tissue samples. See e.g., Sambrook et al. (1989); Perbal (1984); and Scopes (1991).

Total RNA may be extracted by any conventional method known in the art. Total RNA may be extracted, for example, using methods comprising guanidinium hydrochloride and cesium chloride (Glisin et al. (1974) Biochemistry 13: 2633; Ullrich et al. (1977) Science 196: 1313; Chomczynski et al. (1987) Anal. Biochem. 162: 156) or by methods comprising guanidinium hydrochloride and organic solvents (Strohman et al. (1977) Cell 10: 265; McDonald et al. (1987) Meth. Enzymol. 152: 219). Alternatively, RNA extraction kits are commercially available. For example, RNA STAT 60® (Tel-Test, Inc., Friendswood, Tex.), RNeasy® (QIAGEN), Tripure® (Boehringer Mannheim Biochemicals, Indianapolis, Ind.), Trizol (GIBCO Laboratories, Gaithersburg, Md.), and Tri Reagent® (Molecular Research Center, Inc., Cincinnati, Ohio).

The normalization controls of the present invention can be added to the nucleic acid sample from concentrated stock solutions to bring the normalization control to the desired concentration. Preferably, the normalization controls are added to the nucleic acid sample from a 2× stock solution; more preferably from a 10× stock solution; even more preferably from a 20× stock solution; and most preferably from a 100× stock solution.

Without further description, it is believed that one of ordinary skill in the art can, using the preceding description and the following illustrative examples, practice the methods of the present invention. The following working examples therefore, specifically point out the preferred embodiments of the present invention, and are not to be construed as limiting in any way the remainder of the disclosure.

### EXAMPLES

### Example 1

### Preparation of Normalization Control Gene Segments

Clones containing the normalization control genes BioB, BioC, BioD and Cre were obtained from the American Type Culture Collection (ATCC), Manassas, Va. Specifically, pglks-bioB (ATCC 87487), pglks-bioC (ATCC 87488), pglks-bioD (ATCC 87489), pglks-cre (ATCC 87490), and pglbs-dap (ATCC 87486) were used to transform Escherichia coli. The transformed bacterial cells were cultured (50 ml) according to established protocols. The plasmid DNA from the overnight cultures were isolated using QIAGEN® Plasmid Kits and other standard procedures.

Fragments of the normalization control genes, namely normalization control gene segments, were produced and inserted into pBluescript II. The normalization control gene segments were amplified in E. coli, isolated and sequenced. The size and identity of the normalization control gene segments are summarized in Table 1.

TABLE 1

| Control | Insert Size | | Gene |
|---|---|---|---|
| Name | (bp) | Organism | Product |
| BioB 3′ | 350 | E. coil | biotin |
| BioB 5′ | 350 | | synthetase |
| BioB M | 380 | | |
| BioC 3′ | 360 | E. coli | biotin |
| BioC 5′ | 414 | | synthesis |
| | | | protein |
| BioD 3′ | 400 | E. coli | dethiobiotin |
| | | | synthetase |
| Cre 3′ | 503 | P1 phage | site-specific |
| Cre 5′ | 560 | | recombinase |
| Dap 3′ | 667 | B. subtilis | dehydrodipic |
| Dap 5′ | 720 | | olinate |
| Dap M | 665 | | reductase |

10 μg of isolated plasmid DNA containing the normalization control gene segment DapM, Dap5′, Cre5′, BioB3′, BioBM, BioD3′, BioC5′ or Dap3 were digested in a 50 μl reaction volume with XhoI, according to the manufacturer's protocols. BioC3′ and Cre3′ were both linearized with KpnI, which produces a 3′ overhang and thereby prevents the in vitro transcription reaction from continuously producing cRNA of the insert and plasmid. Controls were blunt-ended using T4 polymerase and examined for complete digestion on an E-Gel™ (Invitrogen, Calif.). Digestion of the isolated plasmid was monitored by gel electrophoresis in a 1% agarose gel, using 50 ng each of the uncut and linearized plasmid. Following complete digestion of the isolated plasmid with either XhoI or KpnI, the linearized plasmid DNA was phenol/chloroform/isoamyl alcohol extracted and precipitated with ethanol, according to'established protocols. The linearized plasmid DNA was resuspended in 10 μl of DEPC-treated water and quantified by UV spectrophotometry at 260 nm. The final concentration of the purified DNA (OD 260/280 nm=1.8-2.0) was adjusted to 0.5 μg/μl.

In vitro transcription reactions were performed using 1-2 μg of a normalization control gene segment at 37° C. for 6 hours and Ambion's T7 MegaScript in vitro Transcription Kit. After completion of the reaction, the residual DNA was digested using 1 μl DNase. The cRNA produced was purified by using an RNeasy® Mini Kit (Qiagen, Calif.).

The cRNA was then fragmented (5× fragmentation buffer: 200 mM Tris-Acetate (pH 8.1), 500 mM KOAc, 150 mM MgOAc) for thirty-five minutes at 95° C. The appropriate fragmentation time was determined for each control by subjecting the normalization control gene segment cRNA to fragmentation of varying duration. For example, controls were fragmented between 25 and 50 minutes at 95° C. When the gene segments fragmented at 25, 29, 31, 33, 37, 39, 41, 43, 45 and 56 minutes were run in a PAGE gel, smear decreased with time and decreased most dramatically for the samples fragmented at 33 and 35 minutes. The Average Difference values on the GeneChip™ array platform also decreased with fragmentation time and decreased most dramatically after 33 minutes of fragmentation.

Bio-11-CTP and Bio-16-UTP nucleotides (Enzo Diagnostics) were added to the reaction to biotinylate the cRNA. After a 37° C. incubation for six hours, the labeled cRNA was cleaned up according to the RNeasy Mini kit protocol (QIAGEN).

### Example 2

### Nucleic Acid Sample Acquisition and Preparation.

With minor modifications, the nucleic acid sample preparation protocol followed the Affymetrix GeneChip® Expression Analysis Manual. Frozen tissue was first ground to powder using the Spex Certiprep 6800 Freezer Mill. Total RNA was then extracted using Trizol (Life Technologies). The total RNA yield for each sample (average tissue weight of 300 mg) was about 200-500 μg. Next, mRNA was isolated using the Oligotex mRNA Mini Kit (QIAGEN). Since the mRNA was eluted in a final volume of 400 μl, an ethanol precipitation step was required to bring the concentration to 1 μg/μl. Using 1-5 μg of mRNA, double stranded cDNA was created using the SuperScript Choice system (Gibco-BRL). First strand cDNA synthesis was primed with a T7-(dT₂₄) oligonucleotide. The cDNA was then phenol-chloroform extracted and ethanol precipitated to a final concentration of 1 μg/μl.

55 μg of fragmented cRNA was hybridized on the human 32K set and the HuGeneFL array for twenty-four hours at 60 rpm in a 45° C. hybridization oven, according to the Affymetrix protocol. The chips were washed and stained with Streptavidin Phycoerythrin (SAPE) (Molecular Probes) in Affymetrix fluidics stations. To amplify staining, the chips were washed with SAPE solution, stained with an anti-streptavidin biotinylated antibody (Vector Laboratories) followed by washing with SAPE solution. Hybridization to the probe arrays was detected by fluorometric scanning (Hewlett Packard Gene Array Scanner). Following hybridization and scanning, the microarray images were analyzed for quality control, looking for major chip defects or abnormalities in hybridization signal. After all chips passed quality control, the data was analyzed using Affymetrix GeneChip® software (v3.0), and Experimental Data Mining Tool (EDMT) software (v1.0).

### Example 3

### Cross-Hybridization Analysis of Normalization Controls.

Following fragmentation of the normalization control gene segments, the cRNA were dissolved in MES buffer (101.6 mM MES; 1M NaCl; 0.01% Tween 20; 0.1 mg/ml herring sperm DNA) and the precise concentration for each control was determined. Three dilutions for each control were analyzed —1:200, 1:100 and 1:50. Table 2 shows the three calculated concentrations, the average concentration, the standard deviation (StDev) and the relative standard deviation (RSD). In order to generate consistent normalization control batches, only those controls with RSD less than 6.5% were selected.

TABLE 2

| | 1:200 | | | Avg | | |
|---|---|---|---|---|---|---|
| Control | μg/ml | 1:100 μg/ml | 1:50 μg/ml | μg/ml | StDev | RSD |
| BioB-5′ | 880 | 840 | 840 | 853.3 | 23.1 | 2.71 |
| Dap-M | 480 | 440 | 460 | 460.0 | 20.0 | 4.35 |
| Dap-5′ | 800 | 840 | 900 | 846.7 | 50.3 | 5.94 |
| Cre-5′ | 1040 | 1040 | 1160 | 1080.0 | 69.3 | 6.42 |
| BioB-3′ | 1040 | 960 | 1080 | 1026.7 | 61.1 | 5.95 |
| BioB-M | 1040 | 1080 | 1100 | 1073.3 | 30.6 | 2.85 |
| BioD-3′ | 1040 | 1040 | 1160 | 1080.0 | 69.3 | 6.42 |
| BioC-5′ | 1520 | 1440 | 1520 | 1493.3 | 46.2 | 3.09 |
| BioC-3′ | 640 | 640 | 600 | 626.7 | 23.1 | 3.69 |
| Dap-3′ | 640 | 680 | 700 | 673.3 | 30.6 | 4.54 |
| Cre-3′ | 782 | 800 | 810 | 797.3 | 14.2 | 1.78 |

Fragmented nucleic acid sample alone, or nucleic acid sample mixed with normalization control gene segments was hybridized on the human 32K set and the HuGeneFL array for twenty-four hours at 60 rpm in a 45° C. hybridization oven. The chips were washed and stained with SAPE Solution in Affymetrix fluidics stations. Hybridization to the probe arrays was detected by fluorometric scanning (Hewlett Packard Gene Array Scanner). The cross-hybridization of the candidate normalization control gene segments was analyzed by comparing the binding of the normalization control gene segments in the presence and absence of nucleic acid sample. For example, each normalization control gene segment cRNA was hybridized to a GeneChip® array, in the absence of nucleic acid sample, to confirm that each segment hybridizes to the correct tile or the chip. In addition, nucleic acid sample in the absence of the normalization control cRNA was also hybridized under identical conditions to the GeneChip® array to confirm the absence of cross-hybridization to the normalization control probes on the array.

### Example 4

### Selection of Normalization Controls.

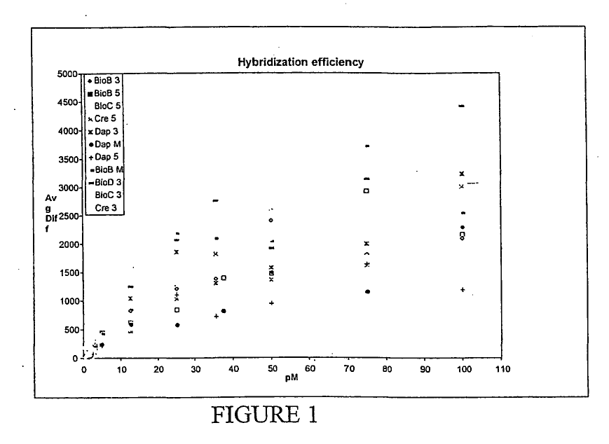

In order to determine the optimal concentration for use with each individual normalization control gene segment, nucleic acid samples were mixed with one concentration for each normalization control gene segment and hybridized to the chip according to the procedures described above. The task of assigning each normalization control gene segment to a specific concentration was complicated by an initial inconsistent performance of the controls. (FIG. 1). To determine the linear performance of each normalization control gene segment and identify the optimal concentration for each control, we hybridized each normalization control gene segment at concentrations ranging from 0.5 to 100 pM. (See, Table 3). Each chip has each control at a different concentration, and the 12 chips assure that each control is measured at the desired concentration. Each normalization control gene segment was analyzed over a range of concentrations that are summarized in Table 3. The median intensity of the normalization control gene segments bound at each concentration was plotted so that the normalization control gene segments that hybridize similarly to the array and produce the most consistently linear curve of hybridization signal over a range of concentrations were selected.

TABLE 3

| Control | | | | | | | | | | | | |
|---|---|---|---|---|---|---|---|---|---|---|---|---|
| Name | chip 1 | chip 2 | chip 3 | chip 4 | chip 5 | chip 6 | chip 7 | chip 8 | chip 9 | chip 10 | chip 11 | chip 12 |
| BioB 5′ | 0.5 | 0.75 | 1 | 1.5 | 2 | 3 | 5 | 12.5 | 25 | 50 | 75 | 100 |
| Dap M | 0.75 | 1 | 1.5 | 2 | 3 | 5 | 12.5 | 25 | 50 | 75 | 100 | 0.5 |
| Dap 5′ | 1 | 1.5 | 2 | 3 | 5 | 12.5 | 25 | 50 | 75 | 100 | 0.5 | 0.75 |
| Cre 5′ | 1.5 | 2 | 3 | 5 | 12.5 | 25 | 50 | 75 | 100 | 0.5 | 0.75 | 1 |
| BioB 3′ | 2 | 3 | 5 | 12.5 | 25 | 50 | 75 | 100 | 0.5 | 0.75 | 1 | 1.5 |
| BioB M | 3 | 5 | 12.5 | 25 | 50 | 75 | 100 | 0.5 | 0.75 | 1 | 1.5 | 2 |
| BioD 3′ | 5 | 12.5 | 25 | 50 | 75 | 100 | 0.5 | 0.75 | 1 | 1.5 | 2 | 3 |
| BioC 5′ | 12.5 | 25 | 50 | 75 | 100 | 0.5 | 0.75 | 1 | 1.5 | 2 | 3 | 5 |
| BioC 3′ | 25 | 50 | 75 | 100 | 0.5 | 0.75 | 1 | 1.5 | 2 | 3 | 5 | 12.5 |
| Dap 3′ | 50 | 75 | 100 | 0.5 | 0.75 | 1 | 1.5 | 2 | 3 | 5 | 12.5 | 25 |
| Cre 3′ | 75 | 100 | 0.5 | 0.75 | 1 | 1.5 | 2 | 3 | 5 | 12.5 | 25 | 50 |

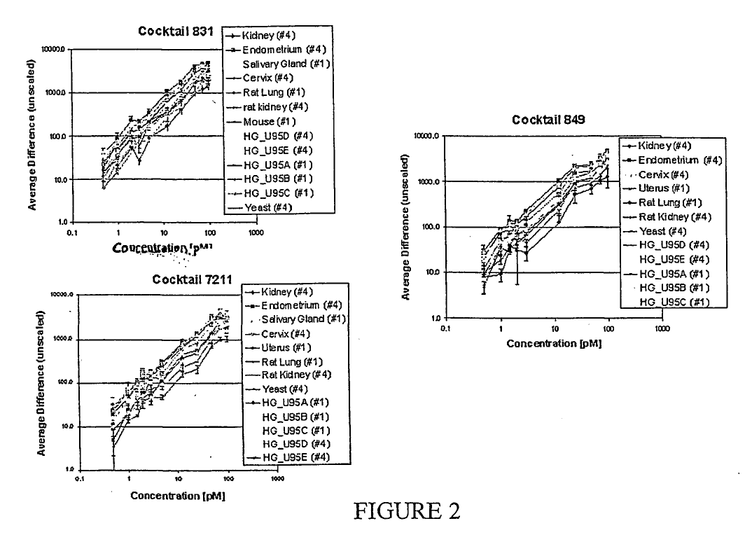

A cocktail of normalization control gene segments at different concentrations is selected based on the linear performance of each cocktail as determined by the linear coefficient (R²) of each cocktail. Specifically, normalization control cocktails, such as those illustrated in Table 3, that display the highest linear performance, based on identifying those cocktails that have the highest average R² and the highest minimum R² values, were selected as normalization controls. In order to minimize the computation time necessary to evaluate all possible normalization control cocktails, the normalization control gene segments BioC5′, Dap3′, and Cre3′ were preassigned to 0.5, 1.0 and 3.0 pM, respectively, based on the linear performance of the individual controls. Furthermore, based on a similar analysis, BioC5′, Dap3′, Dap5′ and DapM each performed best at concentration assignments equal to or below 2 pM, whereas BioB3′ and BioD3′ performed best at either 75 or 100 pM. Three normalization control cocktails were prepared (See, Table 4) and used on various GeneChip arrays. Specifically, cocktails 831, 849, and 7211 were each tested on HG-U95A arrays (5 different tissue types performed in triplicate), rat RG-U34 arrays (2 different tissue types performed in triplicate), Arabidopsis array (one tissue performed in triplicate) and the yeast YG-S98 array (performed in triplicate). The individual standard curves produced for each cocktail on these arrays are presented in FIG. 2.

TABLE 4

| Control | cocktail | cocktail | cocktail |
|---|---|---|---|
| Name | 831 | 849 | 7211 |
| BioB 5′ | 25 | 50 | 12.5 |
| Dap M | 2 | 2 | 2 |
| Dap 5′ | 1.5 | 1.5 | 1 |
| Cre 5′ | 12.5 | 12.5 | 25 |
| BioB 3′ | 100 | 75 | 100 |
| BioB M | 50 | 25 | 50 |
| BioD 3′ | 75 | 100 | 75 |
| BioC 5′ | 1 | 1 | 1.5 |
| BioC 3′ | 3 | 5 | 5 |
| Dap 3′ | 0.5 | 0.5 | 0.5 |
| Cre 3′ | 5 | 3 | 3 |

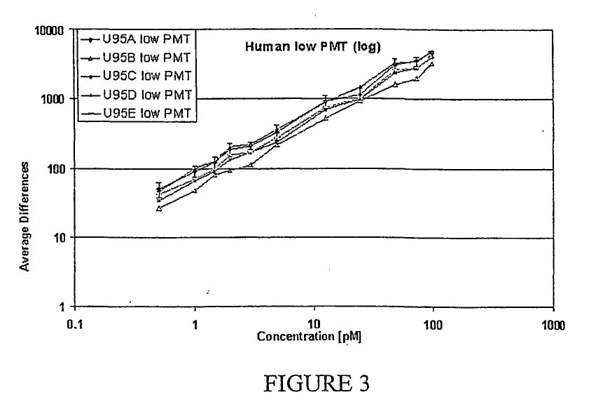

The GeneChip array experiments with normalization control cocktails 831, 849, and 7211 indicate that cocktail 7211 exhibits the highest R². Only at concentrations greater than 50 pM did this normalization control cocktail display nonlinearity, in contrast to cocktail 849. Therefore, BioB3′ was assigned to 75 pM and BioD3′ to 100 pM, respectively, to further improve the linear performance of cocktail 7211. The standard curve based on the improved 7211 cocktail (R^{2=0.985}) is shown in FIG. 3.

Although the present invention has been described in detail with reference to examples above, it is understood that various modifications can be made without departing from the spirit of the invention. Accordingly, the invention is limited only by the following claims. All cited patents and patent applications and publications referred to in this application are herein incorporated by reference in their entirety.

1. A method of normalizing a hybridization reaction comprising a nucleic acid sample, comprising:
a) adding at least one normalization control gene segment to the hybridization reaction corresponding to the 5′, middle or 3′ regions of at least one normalization control gene.

2. A method of claim 1, wherein the normalization control gene segment is not present in the nucleic acid sample.

3. A method of claim 2, wherein the normalization control genes are selected from the group consisting of:
a) viral genes;
b) prokaryotic genes; and
c) eukaryotic genes.

4. A method of claim 2, wherein hybridization reaction is conducted on a solid substrate.

5. A method of claim 4, wherein the solid substrate is an oligonucleotide array.

6. A method of claim 5, wherein the array comprises oligonucleotide probes that are complementary to the normalization control gene segments.

7. A method of claim 6, wherein the oligonucleotide probes of the array are selected from the group consisting of:
a) human nucleic acids;
b) non-human nucleic acids;
c) animal nucleic acids;
d) microbial nucleic acids;
e) bacterial nucleic acids;
f) fungal nucleic acids;
g) tissue specific nucleic acids;
h) disease specific nucleic acids; and
i) plant nucleic acids.

8. The method of claim 7, wherein the normalization control gene segments are selected by a method comprising determining the non-specific cross-hybridization of the nucleic acid sample to the normalization control gene segments.

9. The method of claim 8, wherein the normalization control gene segments that do not substantially cross-hybridize are selected.

10. The method of claim 7, wherein the normalization control gene segments that are added to the hybridization reaction are selected by a method comprising analyzing a series of hybridization reactions wherein each hybridization reaction of the series contains an increased concentration of the normalization control gene segment.

11. The method of claim 10, wherein the normalization control gene segments that produce the most consistently linear curve of hybridization signal over a range of normalization control gene segment concentrations are selected.

12. The method of claim 1, wherein the normalization control gene segments are the 5′, middle and 3′ fragments of at least one normalization control gene.

13. A method of normalizing a hybridization reaction comprising a nucleic acid sample, comprising:
a) providing a normalization control comprising one or more normalization control gene segments, wherein said normalization control gene segments are mixed with the nucleic acid sample, and wherein said normalization control gene segments are prepared by a method comprising:
i) selecting one or more candidate normalization control genes;
ii) segmenting the candidate normalization control genes into 5′-, middle-, and 3′-segments, thereby producing candidate normalization control gene segments;
iii) hybridizing said candidate normalization control gene segments to an oligonucleotide probe in the presence and absence of the nucleic acid sample;
iv) determining the non-specific cross-hybridization of candidate normalization control gene segments to said oligonucleotide probe by determining the hybridization of candidate normalization control gene segments to probes other than those complementary to the candidate normalization control gene segments;
v) repeating step (iii) at various concentrations of candidate normalization control gene segments; and
vi) identifying and selecting those candidate normalization control gene segments that do not substantially cross-hybridize to said oligonucleotide probe.

14. The method of claim 13, wherein the normalization control gene segments are prepared by method further comprising the following steps:
a) preparing individual mixtures of nucleic acid samples and candidate normalization control gene segments wherein each individual mixture contains a different concentration of the candidate normalization control gene segments identified in step (vi);
b) hybridizing a mixture of step (a) to an oligonucleotide probe;
c) repeating step (b) with mixtures containing different concentrations of candidate normalization control gene segments;
d) identifying the candidate normalization control gene segments that produce the most consistently linear hybridization response over a range of candidate normalization control gene segment concentrations by measuring the hybridization of said candidate normalization control gene segments to oligonucleotide probes that are complementary to the normalization control gene segments over a range of candidate normalization control gene segment concentrations; and
e) producing a solution containing one or more of the candidate normalization control gene segments of step (d) over a concentration range sufficient to produce a linear normalization curve.

15. The method of claim 14, further comprising the steps of:
a) hybridizing a mixture of said nucleic acid sample and the solution of step (e) to said array; and
b) quantifying the hybridization of said target or pool of nucleic acid sample to said array.

16. A method of claim 13, wherein the normalization control gene segment is not present in the nucleic acid sample.

17. A method of claim 16, wherein the normalization control genes are selected from the group consisting of:
a) viral genes;
b) prokaryotic genes; and
c) eukaryotic genes.

18. A method of claim 16, wherein hybridization reaction is conducted on a solid substrate.

19. A method of claim 18, wherein the solid substrate is an oligonucleotide array.

20. A method of claim 19, wherein the nucleotide array comprises oligonucleotide probes that are complementary to the normalization control gene segments.

21. A method of claim 20, wherein the oligonucleotide probes of the oligonucleotide array are selected from the group consisting of:
a) human nucleic acids;
b) non-human nucleic acids;
c) animal nucleic acids;
d) microbial nucleic acids;
e) bacterial nucleic acids;
f) fungal nucleic acids;
g) tissue specific nucleic acids;
h) disease specific nucleic acids; and
i) plant nucleic acids.

22. The method of claim 1, wherein the normalization control gene segments are labeled.

23. The method of claim 22, wherein the label is selected from one or more of the group consisting of:
a) a fluorescent label;
b) a chemiluminescent label;
c) a bioluminescent label;
d) a radioactive label;
e) colorimetric label; and
f) a light scattering label.

24. The method of claim 1, wherein the normalization control gene segments are produced by the polymerase chain reaction.

25. The method of claim 1, wherein the normalization control gene segments are produced by cloning into a vector and expressing said normalization control gene segments in a host cell.

26. The method of claim 1, wherein the normalization control gene segments are DNA or RNA.

27. The method of claim 26, wherein the normalization control gene segments are RNA.

28. The method of claim 1, further comprising fragmenting the normalization control gene segments.

29. The method of claim 1, wherein the normalization control genes are selected from one or more of the group consisting of:
a) an Escherichia coli BioB gene;
b) an Escherichia coli BioC gene;
c) an Escherichia coli BioD gene;
d) a P1 bacteriophage Cre gene;
e) a Bacillus subtilis dap gene;
f) a Bacillus subtilis thr gene;
g) a Bacillus subtilis trp gene;
h) a Bacillus subtilis phe gene; and
i) a Bacillus subtilis lys gene.

30. The method of claim 1, wherein the nucleic acid sample is selected from the group consisting of:
a) pooled nucleic acid samples;
b) genomic DNA;
c) cDNA;
d) cRNA;
e) mRNA; and
f) polyA RNA.

31. The method of claim 5, wherein the oligonucleotide probe array is immobilized on a solid support selected from the group consisting of:
a) filters;
b) polyvinyl chloride dishes;
c) silicon or glass beads; and
d) glass wafers.

32. The method of claim 5, wherein the oligonucleotide probe array is a high density array or nucleic acid chip.

33. A method of claim 29, wherein the normalization control genes are selected from the group consisting of BioB, Dap, Cre, BioD, and BioC.

34. A method of claim 29, wherein the normalization control genes consist of BioB, Dap, Cre, BioD, and BioC.

35. A method of claim 34, wherein the normalization control gene segments comprise BioB 5′, Dap M, Dap 5′, Cre 5′, BioB 3′, BioB M, BioD 3′, BioC 5′, BioC 3′, Dap3′ and Cre 3′.

36. A method of claim 34, wherein the normalization control gene segments are a cocktail comprising BioB 5′, Dap M, Dap 5′, Cre 5′, BioB 3′, BioB M, BioD 3′, BioC 5′, BioC 3′, Dap 3′ and Cre 3′.

37. A method of claim 36, wherein the cocktail is cocktail 7211 in FIG. 4.

38. A method of claim 37, wherein the cocktail comprises normalization control gene fragments BioB 5′ at about 12.5 pM, Dap M at about 2 pM, Dap 5′ at about 1 pM, Cre 5′ at about 25 pM, BioB 3′ at about 100 pM, BioB M at about 50 pM, BioD 3′ at about 75 pM, BioC 5′ at about 1.5 pM, BioC 3′ at about 5 pM, Dap 3′ at about 0.5 pM and Cre 3′ at about 3 pM.
